(19) United States
(12) Patent Application Publication
(10) Pub. No.: US 2017/0021043 A1
(43) Pub. Date: Jan. 26, 2017

(54) COUNTING MACHINE-LOADABLE BILL STERILIZER USING ION CLUSTERS

(71) Applicant: SMI CO., LTD., Gangwon-do (KR)

(72) Inventor: Myung Soo BAEK, Gangwon-do (KR)

(73) Assignee: SMI CO., LTD., Gangwon-do (KR)

(21) Appl. No.: 14/965,117

(22) Filed: Dec. 10, 2015

(30) Foreign Application Priority Data
Jul. 23, 2015 (KR) 20-2015-0004961

(51) Int. Cl. A61L 2/14; A61L 2/24; A61L 2/00

(52) U.S. Cl. CPC A61L 2/14; A61L 2/0011; A61L 2/24

(57) ABSTRACT

Disclosed herein is a counting machine-loadable bill sterilizer using ion clusters. The whole structure of the counting machine configured to count the bills is fixedly disposed in the inside of a body configured to disinfect the bills, and cluster anion is supplied into the inside of the body, whereupon the anion can be uniformly transferred onto the surfaces of each bill, thus eliminating any contaminant which might be on the bills.

### TECHNICAL FIELD

The present invention relates to a bill sterilizer, and in particular to a counting machine-loadable bill sterilizer using ion clusters which makes it possible to cleanly and stably sterilize, with the aid of sprayed anion, the bills which are being counted by a counting machine which has been loaded inside the bill sterilizer, while sterilizing user's hands.

### BACKGROUND ART

Money in general is regarded as a basic value means which forms the foundation of an economical structure and has been used for long time in the forms of bills and coins. Different from the coin, the bills may be easily contaminated by various contaminants, for example, bacteria, dirt, etc. during a number of distributions, whereupon the users may be infected by bacteria. The bills are likely to be distributed from hand to hand rather than they are stored long in one place, during which the bills may be contaminated by various bacteria or various pathogenic microorganism. For this reason, the bills may be a medium which can cause or infect various skin diseases and respiratory system diseases as well as allergic diseases. To this end, elderly people or kids who have weak immunity may have any damages due to such diseases, and workers who handle with bills at banks may have the damages.

The banks or the like are using a bill counting machine since it needs to quickly count a lot of bills. Such a bill counting machine is able to count the bills and has a problem sterilizing contaminants, for example, various bacteria, dirt, etc. on the bills.

The above bill counting machine may be categorized into various types. Most of such bill counting machines are configured to count the number of bills using a sensor while the bills loaded in a storing unit are being moved one by one.

The thusly counted bills circulate through markets and are exposed to various contaminants while they are transferred from hand to hand in the markets, whereupon various bacteria can grow on the surfaces of the bills. The thusly contaminated bills may infect multiple people while the bills are being circulated from hand to hand.

In order to resolve the above mentioned problems, a technology has been disclosed, which is able to sterilize bills.

As an example, the Korean patent registration number 10-0423025 whose publication date is Mar. 18, 2004 discloses a bill sterilizer.

The above bill sterilizes includes a shelf-shaped bill support unit on which bills are loaded to count and sterilize the bills; a bill sheet supply device configured to transfer sheet by sheet the bills placed on the bill support unit; a bill transfer device formed of a plurality of rollers which are able to transfer the bills being inputted from the bill sheet supply device and a motor for generating driving force; a sterilization device formed of a spraying device which is able to receive sterilization liquid from a sterilization liquid storing container and spray on the bills in order to sterilize the bills which are being transferred by the bill transfer device; a drying device configured to dry the bills sterilized by the sterilization device; an air freshener supply device configured to spay air freshener onto the bills which have been passed through the drying device; a bill counting device configured to detect the bills while the bills are being transferred by the transfer device and count the passing bills; and a control device formed of a control circuit for controlling the devices and a displayer for indicating the counted number

As another example, the Korean utility model registration number 20-0330878 whose publication date is Feb. 14, 2004 discloses a sterilizer for sterilizing bills which are being counted.

The above sterilizer is directed to a device for sterilizing bills which are being counted, which is able to simultaneously sterilize and disinfect the bills which are being counted.

The above sterilizer includes a bill counting machine; an outer box which is able to accommodate the bill counting machine; a liquid container disposed in the inside of the outer box and filled with disinfection liquid and air freshener; an electronic valve which is able to control the discharge from the liquid container; a nozzle for spraying the discharge liquid; and a connection pipe connected from the liquid container to the nozzle. The nozzle positions at an end of the connection pipe and is formed of a flexible connection pipe the installation position of which may be adjustable for the sake of disinfection before the counted bills are stacked.

In addition, the Korean utility model registration number 20-0464741 whose publication number is Jan. 21, 2013 discloses a bill counting machine with a sterilization function.

In the above bill counting machine with a sterilization function wherein it includes a transfer unit for transferring bills which are inputted into the bill counting machine, a UV sterilization lamp is installed, which is able to sterilize bills.

In the above case, since the disinfection of bills are carried out sheet by sheet, the processing speed is slow, and stable disinfection effects cannot be obtained. Since the device for bill disinfection is complicated, The installation and manufacturing cost a lot.

### PRIOR ART DOCUMENTS

### Patent Documents

(Patent document 0001) Korean patent registration number 10-0423025 whose publication date is Mar. 18, 2004

(Patent document 0002) Korean utility model registration number 20-0330878 whose publication date is Feb. 14, 2004

(Patent document 0003) Korean utility model registration number 20-0464741 whose publication number is Jan. 21, 2013

### DISCLOSURE OF THE INVENTION

Accordingly, the present invention is made in an effort to resolve the above mentioned problems.

It is an object of the present invention to provide a counting machine-loadable bill sterilizer using ion clusters which is able to simultaneously count, sterilize and disinfect bills in such a way that the counting machine is able to count the bills in a space filled with anion.

[symbol]

More specifically, first, the whole structure of the counting machine configured to count the bills is fixedly disposed in the inside of a body configured to disinfect the bills, and cluster anion is supplied into the inside of the body, whereupon the anion can be uniformly transferred onto the surfaces of each bill, thus eliminating any contaminant which might be on the bills.

Second, the user's hands may be disinfected just as the bills are disinfected.

To achieve the above objects,

[symbol]

[symbol]

There is provided a counting machine-loadable bill sterilizer using ion cluster, which may include, but is not limited to, an accommodation space which is formed at one side of a disinfection body and is configured to accommodate a counting machine which is able to count bills; an ion cluster which is provided in the inside of the disinfection body and is able to sterilize and disinfect the bills placed on the counting machine in such a way to emit anion into the accommodation space; a hand disinfection spray unit which is provided at a lower side of the disinfection body and is able to sterilize the surfaces of user's hands in such a way to emit anion from the ion cluster to the outside; an ion guide unit which is installed in the inside of the disinfection body and is configured to guide the anion in such a way that the anion supplied from the ion cluster is supplied into the inside of the accommodation space and in the direction of the hand disinfection unit; and an adjusting unit which is provided on an outer side of the disinfection body and is configured to adjust the generation amount of anion generating from the ion cluster; and a display unit which is provided on an outer side of the disinfection body so as to indicate the amount of generated anion.

There are further provided an opening and closing member which is installed at the hand disinfection spray unit and rotates in accordance with a switching operation and is able to open and close the passage to the hand disinfection spray unit; and a sensor which is disposed on the disinfection body at one side of the hand disinfection spray unit and is configured to output a signal value detected when user's hands are detected, to a control unit and is configured to sense so that the control unit can open the opening and closing member in accordance with the inputted signal value.

The ion guide unit may include, but is not limited to, a plurality of guide holes which are formed on a lower portion of the front side of the disinfection body; a plurality of guide holes which are formed on the bottom surface of the accommodation space and on both the inner walls thereof; a branching unit which is configured to guide the anion generating from an ion cluster into the guide holes formed in the inward direction of the accommodation space through a communication unit; and a hand disinfection spray unit which communicates with the communication unit and is configured to guide the anion into the guide holes on the front side of the disinfection body.

The branching unit may include, but is not limited to, an upward branching unit which is configured to spray the anion in the upward direction from the communication unit and guide the anion toward the guide holes of the accommodation space; and leftward and rightward branching units which are configured to spray upwardly in both the directions from the communication unit and supply the anion from the accommodation space through the guide holes formed on the wall surfaces at both sides of the accommodation space.

In addition, there are further provided a suction fan which is installed at the upward branching unit and is configured to fast supply the anion into the accommodation space, wherein the suction fan is arranged rotatable in leftward and rightward directions; and suction fans which are installed at the leftward and rightward branching units and are configured to fast supply the anion into the accommodation space, wherein the suction fans are arranged rotatable upward and downward facing each other, whereupon the anion can continuously circulate in the inside of the accommodation space in the clockwise direction or the counterclockwise direction about the counting machine.

The accommodation space is opened and closed by a door.

### INDUSTRIAL EFFECTS

The whole structure of the counting machine which is able to count bills is fixedly disposed in the inside of an accommodation space of a disinfection body wherein the bills can be disinfected. Anion is supplied into the body, whereupon anion can be uniformly distributed onto the surface of each bill, thus removing contaminants on the bills.

The anion supplied into the accommodation space of the disinfection body can be discharged in three directions, and angle adjustment is available, whereupon the bills can be disinfected in such a way to circulate anion in the clockwise direction or the counterclockwise direction. Since the anion can be slowly discharged to the outside, disinfection effects can be enhanced, and the anion can be effectively used.

Since the bills and user's hands can be simultaneously disinfected, the bills can be prevented from being re-contaminated.

### BRIEF DESCRIPTION OF THE DRAWINGS

The present invention will become better understood with reference to the accompanying drawings which are given only by way of illustration and thus are not limitative of the present invention, wherein;

FIG. 1 is a perspective view illustrating a procedure where a counting machine is accommodated into a bill sterilizer according to the present invention;

FIG. 2 is a view illustrating a passage where anion circulates when viewing from the front side in FIG. 1;

FIG. 3 is a perspective view illustrating a branching unit configured to guide anion which has been generated from ion clusters in FIG. 2;

FIG. 4 is a lateral cross sectional view for explaining a use state in FIG. 3;

FIG. 5 is a plane cross sectional view for explaining a use state in FIG. 3;

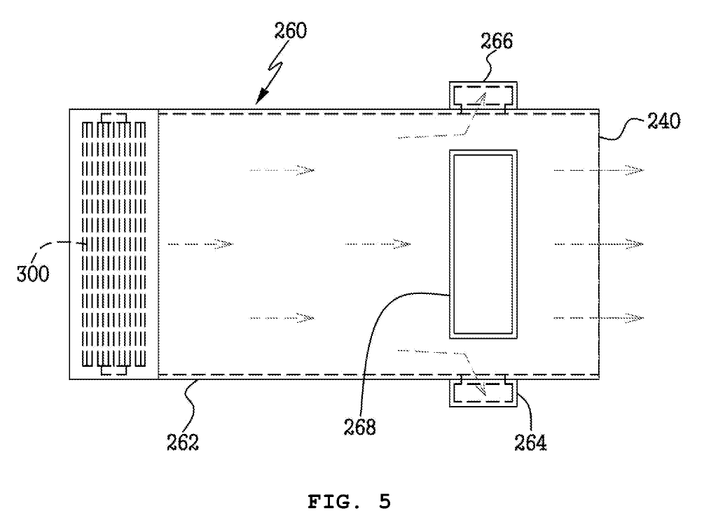

FIG. 6 is a cross sectional view for explaining a use state of an opening and closing member which is disposed at a hand disinfection spray unit in FIG. 3; and

FIG. 7 is a perspective view for explaining a use state where a door is installed at a bill sterilizer in FIG. 1.

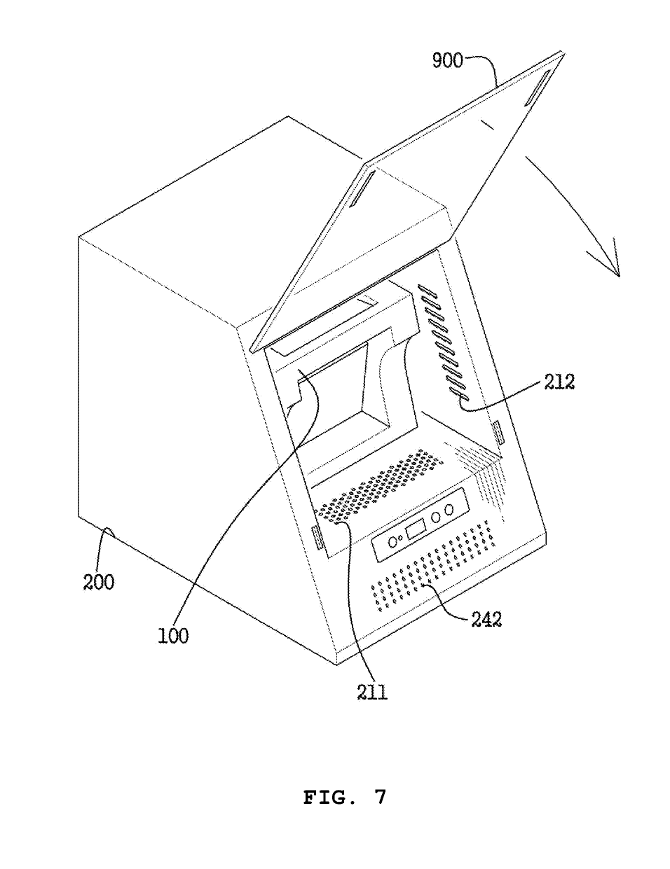

### MODES FOR CARRYING OUT THE INVENTION

The exemplary embodiments of the present invention will be described with reference to the accompanying drawings. In the event that the prior art technology, etc. is assumed to make unclear the subject matter of the present invention, the detailed descriptions thereof will be omitted.

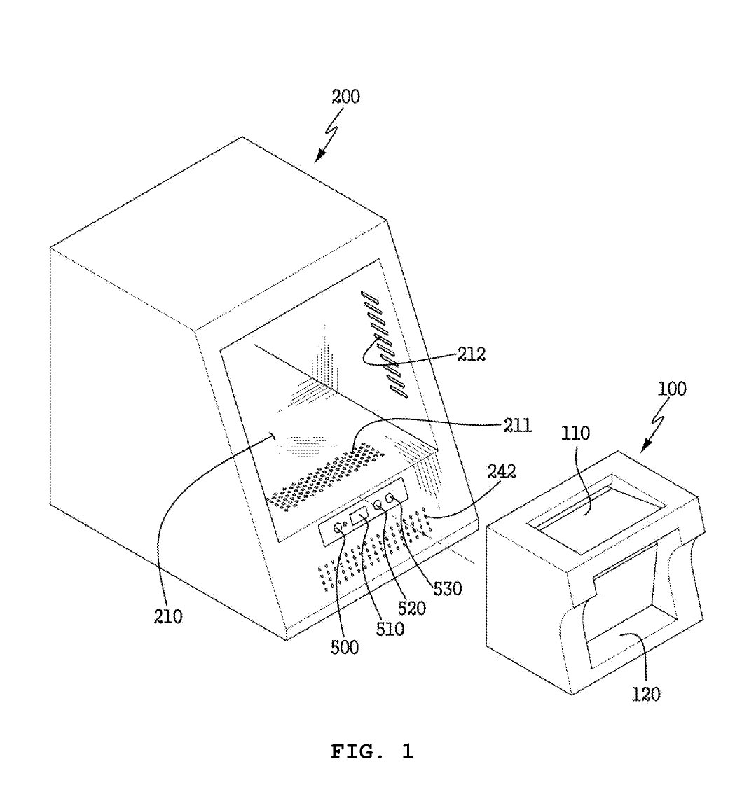

When sterilizing the bills as illustrated in FIG. 1, the counting machine 100 configured to count the bills is accommodated in the inside of the disinfection body 200 which defines the bill sterilizer, thus disinfecting the bills with anion which circulates in the inside.

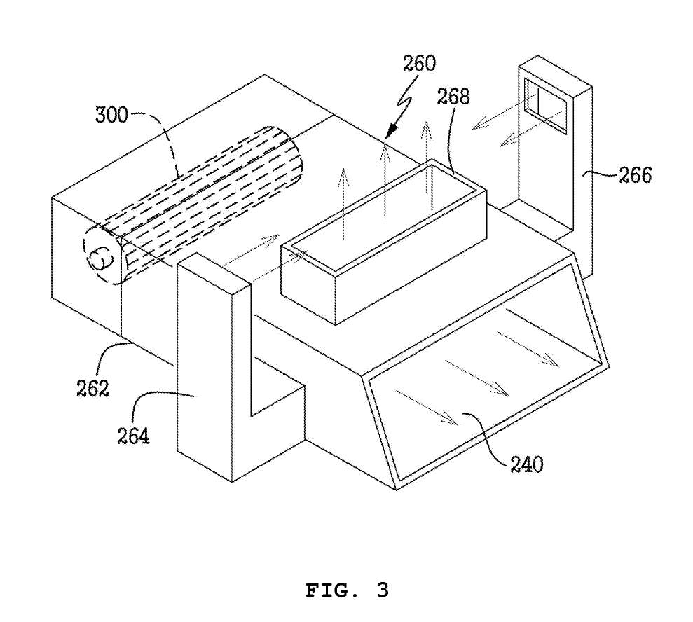

As illustrated in FIGS. 1 to 3, the bill sterilizer of the present invention may include, but is not limited to, a counting machine 100; a disinfection body 200 accommodating the counting machine 100; and an ion cluster 300 emitting anion from the inside of the disinfection body 200. The bills in the counting machine 100 can be sterilized and disinfected in the inside of the disinfection body 200 with the aid of anions generating from the ion cluster 300.

The counting machine 100 may be configured with a conventional technology. When the bills are placed, the counting machine 100 counts the bills and indicates the number of the bills. The counting machine 100 may equip with a bill input unit 110 on its outer top, and a bill discharge unit 120 at a lower portion of its front side.

To this end, the bills inputted through the bill input unit 110 are counted by the counting system, and the bills are discharged through the bill discharge unit 120, and the counted number of the bills is indicated outside.

The disinfection body 200 may include an accommodation space 210 open in one direction at one side of which the counting machine 100 counting the number of the bills is accommodated. Guide holes 211 and 212 through which anion passes are formed on the bottom surface in the inner wall of the accommodation space 210 and on left and right wall surfaces. On a ceiling surface, a sterilization lamp 220 is provided, which is able to sterilize the surfaces of each bill with the emission of infrared ray in the direction of the bill input unit 110 of the counting machine 100 when electric power is supplied.

The ion cluster 300 may formed of any of known anion cluster, plasma anion cluster, anion plasma cluster, etc. The above ion cluster 300 may allow to sterilize and disinfect the bills placed in the counting machine 100 in such a way to emit anion from the inner lower side of the disinfection body 200 into the inside of the accommodation space 210 when electric power is supplied. The ion cluster 300 may include an electric power supply unit; an anion generation unit which generates anion when electric power is supplied; and an anion discharge unit which is able to emit the generated anion into the inside of the accommodation space.

The anion generation unit and the anion discharge unit may be integrated into one body and is able to generate anion when electric power is supplied to the electric power supply unit, and the generated anion moves through the anion discharge unit.

In addition, at a lower front side of the disinfection body 200, a hand disinfection spray unit 240 is provided, which may allow to spray anion generated from the ion cluster 300 to the outside through the guide holes 242. When the anion is discharged through the guide holes 42 communicating with the hand disinfection spray unit 240, the user outside may sterilize his hands.

In the inside of the disinfection body 200, there is provided an ion guide unit 260 which is able to guide the anion generating from the ion cluster 300 into the inside of the accommodation space 210 or toward the hand disinfection spray unit 240.

The ion guide unit 260 may be provided integral with or separate from the disinfection body 200 and may include, but is not limited to, a communication unit 262 communicating with the anion discharge unit of the ion cluster 300; and a plurality of branching units 264, 266 and 268 which communicate with the communication unit 262 and are configured to guide anion onto the bottom surface of the accommodation space 210 and toward both the inner walls.

More specifically, the anion guide unit 260 may include, but is not limited to, an upward branching unit 268 which is formed protruding upward from the communication unit 262 and is able to supply anion through the guide ole 211 formed on the bottom surface of the accommodation space 210; and the leftward and rightward branching units 264 and 266 which are able to supply anion from both the wall surfaces of the accommodation space 210 through the guide holes 212 formed in the direction of the counting machine 100.

To this end, the anion supplied from the ion cluster 300 may be supplied through the communication unit 262 of the ion guide unit 260 and the upward and leftward and rightward branching units 264, 266 and 268 into the guide holes 211 and 211 formed in the three directions at the inner side of the accommodation space 210 and at the same time may be supplied in the direction of the guide hole 242 communicating with the hand disinfection spray unit 240.

The anion supplied to the hand disinfection spray unit 240 is discharged to the outside through the guide holes 242 formed on the front lower side of the disinfection body 200, so the user's hands can be sterilized.

In the present invention, an adjusting unit 500 is provided at a front upper side of the disinfection body 200. The adjusting unit 500 may adjust the generation amount of anion which generates and emits from the ion cluster 300 based on an adjusting value. At one side of the adjusting unit 500, there is provided a display unit 520 which is able to display the amount of anion generating at one side of the adjusting unit 500 in the form of character or graph.

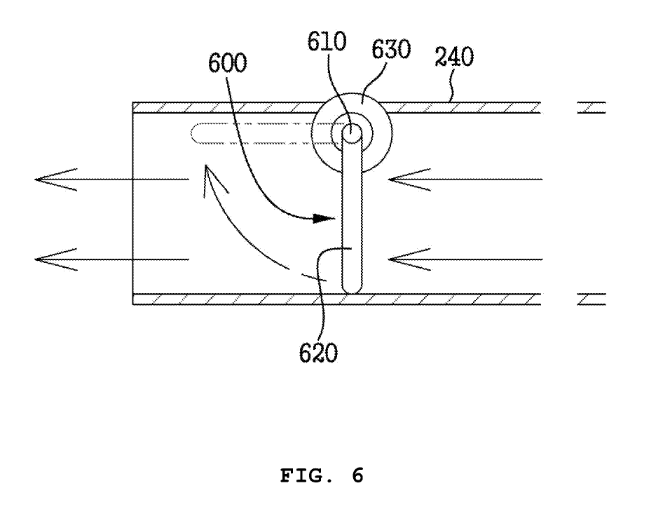

FIG. 6 is a view illustrating another example of the present invention. There is provided an opening and closing member 600 which is opened or closed on the hand disinfection spray unit 240 communicating with the communication unit 262 of the ion guide unit 260 in accordance with a switching operation.

The opening and closing member 600 may allow open or close the hand disinfection spray unit 240 in accordance with a user's selection, whereupon the user can sterilize his hands.

A sensor 530 may be provided at the disinfection body 200 at one side of the hand disinfection spray unit 240. The sensor 530 may output a signal value which generates when the user's hands are detected, to the control unit, and it is preferred that the control unit may allow the opening and closing member 600 to operate in accordance with the inputted signal value.

The opening and closing unit 600 may include, but is not limited to, a rotary shaft 610 both ends of which are engaged rotatable to the inner wall of the hand disinfection spray unit 240; an opening and closing plate 620 which rotates engaged to the rotary shaft 610 and is able to open and close the hand disinfection spray unit 240; and a motor 630 which is engaged to the disinfection body 200 and is able to rotate in a normal or reverse direction the rotary shaft 610.

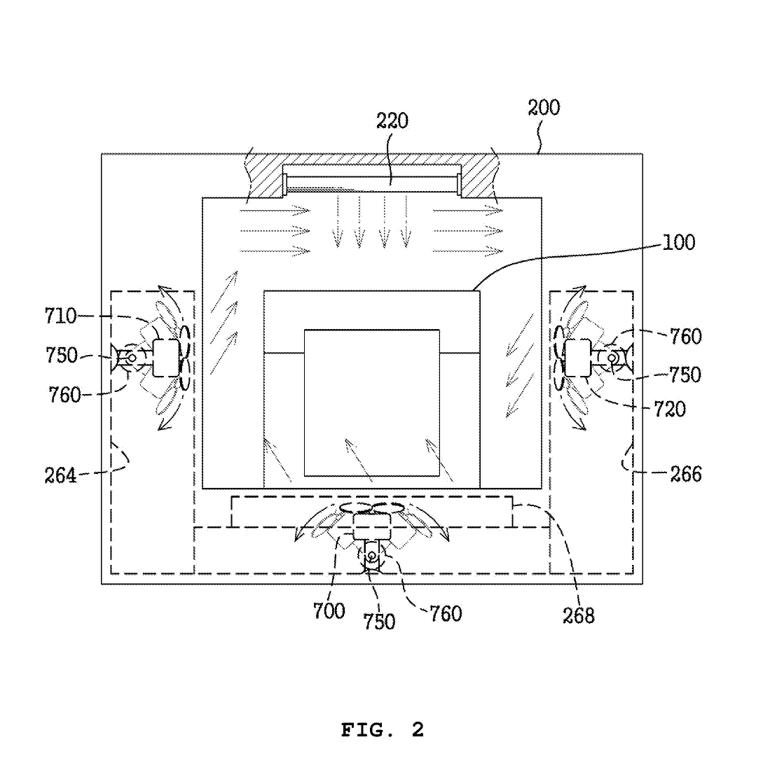

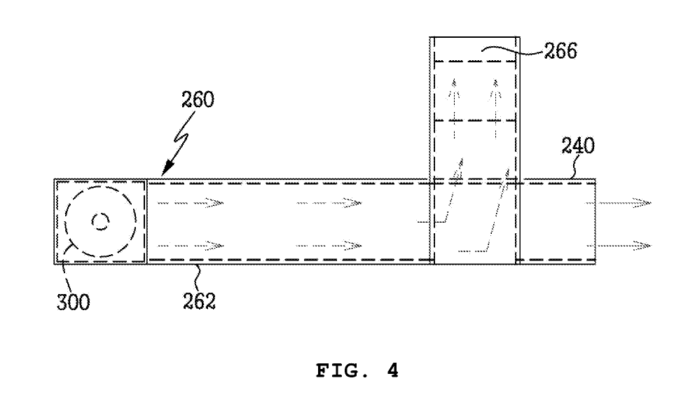

As another example, as illustrated in FIGS. 2 to 4, it is preferred that suction fans 700, 710 and 720 may be installed at the upward branching unit 268, the leftward branching unit 264 and the rightward branching unit 266. The suction fans 700, 710 and 720 may allow to fast supply the anion into the accommodation space 210, which has been generated from the ion cluster 300 during rotation.

Among the above suction fans, the suction fan 700 provided at the upward branching unit 268 may have a rotation angle which can be adjusted in left and right directions, and the suction fans 710 and 720 of the leftward and right branching units 264 and 266 may have rotation angles which can be automatically adjusted in upward and downward directions. With the aid of the rotation angles of the suction fans 700, 710 and 720, the anion may circulate in the above rotation directions in the inside of the accommodation space 210.

The angles of the suction fans 700, 710 and 720 can be adjusted so that the anion can circulate in the directions of the bill input unit 110 and the bill discharge unit 120 of the counting machine 100.

The rotation angles of the suction fans 700, 710 and 720 may be adjusted in accordance with an operation of a remote controller or a mode conversion switch. It is preferred that the mode conversion switch (not illustrated) may be provided at one side of an adjusting unit 500 disposed at an outer side of the disinfection body 200.

As the present invention may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

### LEGEND OF REFERENCE NUMBERS

100: Counting machine
200: Disinfection body
300: Ion cluster
500: Adjusting unit
600: Opening and closing member
610: Rotary shaft
620: Opening and closing plate
630: Motor
700, 710, 720: Suction fan

What is claimed is:

1. A counting machine-loadable bill sterilizer using ion cluster, comprising:
an accommodation space 210 which is formed at one side of a disinfection body 200 and is configured to accommodate a counting machine 100 which is able to count bills;
an ion cluster 300 which is provided in the inside of the disinfection body 200 and is able to sterilize and disinfect the bills placed on the counting machine 100 in such a way to emit anion into the accommodation space 210;
a hand disinfection spray unit 240 which is provided at a lower side of the disinfection body 200 and is able to sterilize the surfaces of user's hands in such a way to emit anion from the ion cluster 300 to the outside;
an ion guide unit 260 which is installed in the inside of the disinfection body 200 and is configured to guide the anion in such a way that the anion supplied from the ion cluster 300 is supplied into the inside of the accommodation space 210 and in the direction of the hand disinfection unit 240; and
an adjusting unit 500 which is provided on an outer side of the disinfection body 200 and is configured to adjust the generation amount of anion generating from the ion cluster 300; and
a display unit 510 which is provided on an outer side of the disinfection body 200 so as to indicate the amount of generated anion.

2. The sterilizer of claim 1, further comprising:
an opening and closing member 600 which is installed at the hand disinfection spray unit 240 and rotates in accordance with a switching operation and is able to open and close the passage to the hand disinfection spray unit 240; and
a sensor 530 which is disposed on the disinfection body 200 at one side of the hand disinfection spray unit 240 and is configured to output a signal value detected when user's hands are detected, to a control unit and is configured to sense so that the control unit can open the opening and closing member in accordance with the inputted signal value.

3. The sterilizer of claim 1, wherein the ion guide unit 260 includes:
a plurality of guide holes 211 which are formed on a lower portion of the front side of the disinfection body 200;
a plurality of guide holes 212 which are formed on the bottom surface of the accommodation space 210 and on both the inner walls thereof;
a branching unit which is configured to guide the anion generating from an ion cluster 300 into the guide holes 211 and 212 formed in the inward direction of the accommodation space 210 through a communication unit 262; and
a hand disinfection spray unit 240 which communicates with the communication unit 262 and is configured to guide the anion into the guide holes 242 on the front side of the disinfection body 200.

4. The sterilizer of claim 3, wherein the branching unit includes:
an upward branching unit 268 which is configured to spray the anion in the upward direction from the communication unit 262 and guide the anion toward the guide holes 211 of the accommodation space 210; and
leftward and rightward branching units 264 and 266 which are configured to spray upwardly in both the directions from the communication unit 262 and supply the anion from the accommodation space 210 through the guide holes 212 formed on the wall surfaces at both sides of the accommodation space 210.

5. The sterilizer of claim 4, further comprising:
a suction fan 700 which is installed at the upward branching unit 268 and is configured to fast supply the anion into the accommodation space 210, wherein the suction fan 700 is arranged rotatable in leftward and rightward directions; and
suction fans 710 and 720 which are installed at the leftward and rightward branching units 264 and 266 and are configured to fast supply the anion into the accommodation space 210, wherein the suction fans 710 and 720 are arranged rotatable upward and downward facing each other, whereupon the anion can continuously circulate in the inside of the accommodation space 210 in the clockwise direction or the counterclockwise direction about the counting machine 100.

6. The sterilizer of claim 5, wherein the accommodation space 210 is opened and closed by a door 900.
